(19) United States
(12) Patent Application Publication
(10) Pub. No.: US 2007/0072228 A1
(43) Pub. Date: Mar. 29, 2007

(54) ERCC2 polymorphisms

(76) Inventor: Hiltrud Brauch, Stuttgart (DE)

Correspondence Address: JENKINS, WILSON, TAYLOR & HUNT, P. A., 3100 TOWER BLVD, SUITE 1200, DURHAM, NC, 27707, US

(21) Appl. No.: 11/529,542

(22) Filed: Sep. 28, 2006

Related U.S. Application Data
(63) Continuation of application No. PCT/EP05/03736, filed on Apr. 8, 2005.
(60) Provisional application No. 60/560,657, filed on Apr. 8, 2004.

(51) Int. Cl. C12Q 1/68; G01N 33/574; C07H 21/04; C07K 16/30

(52) U.S. Cl. 435/6; 435/7.23; 530/388.8; 536/24.3

(57) ABSTRACT

This invention relates to diagnostic methods based upon a polymorphism in individuals indicative of an increased risk of breast carcinoma. More specifically, this invention relates to a method for diagnosis of an increased risk of breast carcinoma by screening for the presence of genetic polymorphisms in individuals, specifically in the ERCC2 gene. The invention is further directed to a method of screening to identify compounds which stimulate the action of a DNA repair enzyme encoded by one of the polymorphic forms of the ERCC2 gene.

### RELATED APPLICATIONS

This application is a continuation of PCT International Patent Application No. PCT/EP2005/003736, filed Apr. 8, 2005, which claims priority to U.S. Provisional Patent Application No. 60/560,657, filed Apr. 8, 2004, the disclosures of each of which are incorporated herein by reference in their entirety.

This invention relates to diagnostic methods based upon a polymorphism in individuals indicative of an increased risk of breast carcinoma. More specifically, this invention relates to a method for diagnosis of an increased risk of breast carcinoma by screening for the presence of genetic polymorphisms in individuals, specifically in the ERCC2 gene. The invention is further directed to a method of screening to identify compounds which stimulate the action of a DNA repair enzyme encoded by one of the polymorphic forms of the ERCC2 gene.

Breast cancer is viewed as a polygenic disease (1), since known susceptibility genes for hereditary breast cancer cannot explain the high breast cancer incidence in western countries. Moreover, genetic models showed that susceptibility to breast cancer is likely to be conferred by a large number of alleles (1). To explore this polygenic nature association studies have become popular. Other than the previous mendelian inheritance approach for the identification of single but uncommon predisposing genes association approaches pay attention to the range of genetic variation across many loci in the population in order to test their predictive value for defining risk groups. Accordingly, breast cancer risk will be estimated from a combined effect of genetic variations. Critical to this approach are an evidence based selection of genetic variants to be tested for eligibility as risk factors, and the avoidance of major selection bias in the study population subjected to analysis.

Women with breast cancer have been shown to have significantly reduced DNA repair proficiencies (2). This finding calls attention to the intricate network of DNA repair systems that protect the genome from deleterious endogenous and exogenous DNA damage (3). In particular enzymes of the nucleotide excision repair (NER) pathway are known or suspected to be implicated in cancer. Importantly, they may also participate in other regulatory cellular processes including DNA replication and basal transcription (4), cell cycle progression (5) and apoptosis (6). The DNA helicase encoded by the excision repair cross-complementing group 2 gene (ERCC2, formerly XPD) is one of seven NER enzymes that cause xerodemma pigmentosum (XP) when mutated in the germline (7). XP is a rare autosomal recessive disease characterized by an extreme sensitivity to sunlight and a greater than 1000-fold increased risk of skin cancer (8). Based on its multiple cellular functions and on rare ERCC2 mutations giving rise to genetic disease, ERCC2 polymorphisms such as ERCC2_{—}6540_G>A and ERCC2_{—}18880_A>C may operate as cancer susceptibility factors (9). ERCC2_{—}6540_G>A (rs1799793), located in exon 10, is implicated in an amino acid exchange from aspartic acid (Asp) to asparagine (Asn) in position 312. This residue is located in the seven-motif helicase domain of the RecQ family of DNA helicases and evolutionarily highly conserved, a reason why this substitution may be of functional significance (10). ERCC2_{—}18880_A>C (rs1052559), located in exon 23, is implicated in an amino acid exchange from lysine (Lys) to glutamine (Gln) in position 751.

The current body of literature supplies conflicting data on the role of ERCC2 polymorphisms and the risk for cancers including glioma (11), melanoma (12), basal cell carcinoma (13), bladder (14), lung (4, 10, 15-21), prostate (22), head and neck (23) as well as breast cancer (9, 24). Supportive evidence for a role of ERCC2 polymorphisms in breast cancer comes from observations of an association of the genotype encoding Gln/Gln at position 751 and increased polycyclic aromatic hydrocarbon (PAH) adduct levels in tumor tissue (24). Also the genotype encoding Lys/Lys at position 751 was associated with reduced DNA repair capacity in lymphocytes of breast cancer patients (9).

Recently, a review of epidemiological studies assessing associations between DNA repair polymorphisms and the risk of cancer provided substantial criticism with respect to the study design of association studies (25). Evaluation of 30 studies showed consistent data only for three out of 29 polymorphisms in three of 8 DNA repair genes. Suggestive results were seen for others however, it was suggested that small sample sizes may have contributed to false-positive or false-negative findings. Altogether, it has been recommended that informative and reliable association studies must be large, favorably greater than 500 cases and controls as well as population-based, and that well designed studies of common polymorphisms in DNA repair are needed to clarify their role in cancer (25).

As a summary, it can be said that the prior art which is at hand today presents an inconsistent image of known ERCC2 polymorphisms and their potential influence on several types of cancer, in particular breast cancer.

Therefore, it is an object of the present invention to provide a method for diagnosis of an increased risk of breast cancer, which offers a precise determination of an individuals risk.

It is a further object of this invention to provide a method of identifying an individual at an increased risk of breast carcinoma associated with a polymorphism in the ERCC2 gene.

It is a still further object to provide screening assays to identify compounds for use in the treatment of breast cancer.

Additionally, it is a still further object to provide a method of treating patients with an increased risk or predisposition to breast carcinoma.

These objects are solved by the subject-matter of the independent claims. Preferred embodiments are set forth in the dependent claims.

To elucidate the role of ERCC2 polymorphisms in breast cancer, the inventors performed a large population-based association study in Germany and provided evidence for a breast cancer predictive role of ERCC2 genotypes and haplotypes.

As mentioned above, the polygenic concept of breast cancer susceptibility calls for the identification of genetic variants contributing to breast cancer risk. The nucleotide excision repair (NER) enzyme encoded by the excision repair cross-complementing group 2 gene ERCC2 (formerly XPD) known to cause skin cancer by germline mutations has multiple regulatory cellular functions including NER, basal transcription, cell cycle control and apoptosis. ERCC2 polymorphisms ERCC2_{—}6540_G>A (Asp312Asn) and ERCC2_{—}18880_A>C (Lys751Gln) within the coding region of this evolutionarily highly conserved gene are shown herein to be of functional relevance, and therefore be candidates to confer breast cancer susceptibility.

Using MALDI-TOF MS methodology the inventors analyzed genotype frequencies in constitutional DNA of study participants of a German case-control study including 688 incident breast cancer cases and 724 population-based, age-matched controls. We identified ERCC2_{—}6540_GG (Asp312Asp) as an at-risk genotype (OR 2.13; 95% CI: 1.44-3.16). The ERCC2_{—}6540_GG associated breast cancer risk was even higher in women who were also carriers of the ERCC2_{—}18880_CC (Gln751Gln) (OR=5.51, 95% CI: 2.37-12.81) or ERCC2_{—}18880_AC (Lys751Gln) genotype (OR=3.70, 95% CI: 2.09-6.53). We identified ERCC2_{—}6540_G/ERCC2_{—}18880_C (312Asp751Gln) as the most potent risk conferring haplotype (OR 3.30, 95% CI 2.47-4.41).

Herein, assigning breast cancer risk to both the ERCC2 genotype encoding Asp312Asp and the haplotype encoding 312Asp/751Gln is done for the first time.

The present invention is directed to the following aspects and embodiments:

According to a first aspect, the invention provides a method of identifying an individual at an increased risk of cancer associated with a polymorphism in a gene, comprising
a) providing a sample from the individual containing genomic DNA;
b) determining the genotype of the ERCC2 gene of the individual;
c) identifying polymorphisms on the ERCC2 gene associated with the predisposition or susceptibility to cancer;
d) assessing the predisposition or susceptibility to cancer of the individual.

The above referenced method is an ex vivo method. Or in other words, it actually comprises the steps of determining the genotype of the ERCC2 gene of the individual in a sample from the individual containing genomic DNA, identifying polymorphisms on the ERCC2 gene associated with the predisposition or susceptibility to cancer and assessing the predisposition or susceptibility to cancer of the individual.

The sample taken from the individual is preferably a blood sample, which is more preferably a heparinized blood sample. However, it should be clear that any kind of sample taken from the individual's body can be used in the method of the present invention, under the provisio that the sample contains genomic DNA.

It is noted that the present invention is preferably directed to the determination of breast cancer risk, may, however, also be used in determining risks related to other types of cancer, including, but not limited to, glioma, melanoma, basal cell carcinoma, bladder, lung, prostate, head and neck cancer.

According to a preferred embodiment, a polymorphism on the ERCC2 gene is at nucleotide position 6540 of the genomic sequence. This nucleotide position corresponds to amino acid position 312 located in exon 10 of the ERCC2 gene. For an explanation regarding the nucleotide position, see also chapter Examples.

According to a further preferred embodiment, a polymorphism on the ERCC2 gene is at nucleotide position 18880 of the genomic sequence. This nucleotide position corresponds to amino acid position 751 located in exon 23 of the ERCC2 gene.

It is explicitly noted that the present invention is in particular directed to the use of both polymorphisms, i.e. 6540 and 18880 in the methods disclosed herein. It surprisingly turned out that an excellent prediction of predisposition or susceptibility to breast cancer can be made by determining and classifying both the ERCC2 genotype at positions 6540 and 18880. The results will be provided below as well as in chapter Examples.

According to one embodiment, the polymorphism at position 6540 is AA, GA or GG. To give an explanation on the results of this polymorphism on the amino acid level, the following is noted:

This is the ERCC2 polymorphism ERCC2_{—}6540_G>A (Asp312Asn). It means that an alteration in the genotype occurs from GAC coding for Asp to AAC coding for Asn. AA, as an example, means that both alleles of an individual is coding Asn, whereas GA means that one allele is coding for Asp, the other for Asn, etc.

According to a further preferred embodiment, the polymorphism at position 18880 is CC, AC or AA.

Corresponding to the explanation given above, this is the ERCC2 polymorphism ERCC2_{—}18880_A>C (Lys751Gln). This means that an alteration in the genotype occurs from AAG coding for Lys to CAG coding for Gln. CC, as an example, means that both alleles of an individual are coding for Gln, whereas AC means that one allele is coding for Lys, the other for Gln, etc.

It surprisingly turned out that the predisposition or susceptibility to breast cancer is highest for GG regarding the polymorphism at position 6540. The precise risk evaluation can be taken from Tables 3 and 4. From Table 3 it can be seen that the OR for an individual is 2,13 for GG. OR (odds ratio) is defined as follows:

The OR is a comparison of the presence of a risk factor for disease in a sample of diseased subjects and non diseased controls. The number of people with disease who were exposed to a risk factor (Ie) over those with disease who were not exposed (Io) divided by those without disease who were exposed (Ne) over those without disease who were not exposed (No). Thus OR=(Ie/Io)/(Ne/No)=Ie No/Io Ne.

The exposed proportion will be higher among the diseased than among the undiseased if the odds ratio will be greater than 1.0. This indicates a positive association between exposure and disease. An odds ratio less than 1 indicates a negative association (protective effect).

According to a further embodiment the predisposition or susceptibility to breast cancer is lowest for AA at position 6540. Generally, the predisposition or susceptibility to breast cancer is increasing in the following order and can be classified as follows: AA<GA<GG (see also Table 3).

Analogously, the predisposition or susceptibility to breast cancer is highest for CC and lowest for AA at position 18880. The predisposition or susceptibility to breast cancer at position 18880 is increasing in the following order: AA<AC<CC (see also Table 3).

As mentioned above, it surprisingly turned out that the predisposition or susceptibility to breast cancer is highest for: GG+CC, the first genotype being at nucleotide position 6540, the second at nucleotide position 18880. This surprising effect is precisely indicated in Table 4 enclosed herewith. OR is on average 5,51 for this combination meaning a remarkably and significantly increased risk of developing breast cancer.

It furthermore turned out that the predisposition or susceptibility to breast cancer is lowest for AA+AC, the first genotype being at nucleotide position 6540, the second at nucleotide position 18880.

The predisposition or susceptibility to breast cancer preferably is increasing in the following order:

group a) AA+AC<GA+AC<AA+AA

group b) AA+CC<GA+AA<GG+AA

group c) GA+CC<GG+AC<GG+CC, the first genotype of each pair being at nucleotide position 6540, the second at nucleotide position 18880.

More preferably, the predisposition or susceptibility to breast cancer preferably is increasing in the order: group a)<group b)<group c).

Having regard to the haplotype of the individuals, it is also predictive for the predisposition or susceptibility to breast cancer (see Table 5). The highest risk exists for haplotype GC, followed by GA, AC and AA (1^{st} letter=pos. 6540, 2^{nd} letter=pos. 18880).

The genotyping in step b) of the method of the invention can be done, for example by using common approaches as PCR as indicated in the Examples or by using specific antibodies, for example monoclonal antibodies, directed against the specific polymorphic amino acid sequences.

The antibody is preferably selected from a group, which consists of polyclonal antibodies, monoclonal antibodies, humanized antibodies, chimeric antibodies and synthetic antibodies.

The antibody according to the invention can be additionally linked to a toxic and/or a detectable agent.

The term “antibody”, is used herein for intact antibodies as well as antibody fragments, which have a certain ability to selectively bind to an epitop. Such fragments include, without limitations, Fab, F(ab′)₂ und Fv antibody fragment. The term “epitop” means any antigen determinant of an antigen, to which the paratop of an antibody can bind. Epitop determinants usually consist of chemically active surface groups of molecules (e.g. amino acid or sugar residues) and usually display a three-dimensional structure as well as specific physical properties.

The antibodies according to the invention can be produced according to any known procedure. For example the pure complete protein according to the invention or a part of it can be produced and used as immunogen, to immunize an animal and to produce specific antibodies.

The production of polyclonal antibodies is commonly known. Detailed protocols can be found for example in Green et al, Production of Polyclonal Antisera, in Immunochemical Protocols (Manson, editor), pages 1-5 (Humana Press 1992) und Coligan et al, Production of Polyclonal Antisera in Rabbits, Rats, Mice and Hamsters, in Current Protocols In Immunology, section 2.4.1 (1992). In addition, the expert is familiar with several techniques regarding the purification and concentration of polyclonal antibodies, as well as of monoclonal antibodies (Coligan et al, Unit 9, Current Protocols in Immunology, Wiley Interscience, 1994).

The production of monoclonal antibodies is as well commonly known. Examples include the hybridoma method (Kohler and Milstein, 1975, Nature, 256:495-497, Coligan et al., section 2.5.1-2.6.7; and Harlow et al., Antibodies: A Laboratory Manual, page 726 (Cold Spring Harbor Pub. 1988).), the trioma technique, the human B-cell hybridoma technique (Kozbor et al., 1983, Immunology Today 4:72), and the EBV-hybridoma technique to produce human monoclonal antibodies (Cole, et al., 1985, in Monoclonal Antibodies and Cancer Therapy, Alan R. Liss, Inc., pp. 77-96).

In brief, monoclonal antibodies can be attained by injecting a mixture which contains the protein according to the invention into mice. The antibody production in the mice is checked via a serum probe. In the case of a sufficient antibody titer, the mouse is sacrificed and the spleen is removed to isolate B-cells. The B cells are fused with myeloma cells resulting in hybridomas. The hybridomas are cloned and the clones are analyzed. Positive clones which contain a monoclonal antibody against the protein are selected and the antibodies are isolated from the hybridoma cultures. There are many well established techniques to isolate and purify monoclonal antibodies. Such techniques include affinity chromatography with protein A sepharose, size-exclusion chromatography and ion exchange chromatography. Also see for example, Coligan et al., section 2.7.1-2.7.12 and section “Immunglobulin G (IgG)”, in Methods In Molecular Biology, volume 10, pages 79-104 (Humana Press 1992).

According to a further aspect, the present invention is directed to a method of screening to identify compounds which modulate the apoptotic capacity of cells in an individual, comprising:

providing cells, in particular breast epithelial cells, of an individual genotype as explained above,

contacting the cells with a candidate compound,

observing the time course of apoptotic response of said cells, and

isolating those compounds, which are agonists of a higher apoptotic response.

According to a further aspect, the invention is directed to an agonist identified by the method as defined above.

According to a still further aspect, a method of treating patients is provided by the present invention comprising identifying a patient with a predisposition to breast cancer by identifying polymorphisms in an ERCC2 gene associated with breast cancer as disclosed above and administering to such patient an effective amount of an agonist identified as above in a pharmaceutically acceptable carrier. Such a pharmaceutical composition may comprise a therapeutically-effective amount of one or more of the agonists identified as above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin.

The phrase “therapeutically-effective amount” as used herein means that amount of an agonist which is effective for producing some desired therapeutic effect by inhibiting the proliferation and/or inducing the differentiation of at least a sub-population of cells in an animal at a reasonable benefit/risk ratio applicable to any medical treatment.

The phrase “pharmaceutically acceptable” is employed herein to refer to those materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The formulations of the present invention may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, each containing a predetermined amount of an agonist as an active ingredient. A compound may also be administered as a bolus, electuary or paste.

In solid dosage forms for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof, and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent.

Tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the agonist may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of this invention suitable for parenteral administration comprise one or more agonists in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the agonist in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

The invention is also comprising the use of an agonist disclosed above in a method of treating breast cancer.

According to a final aspect, the present invention is directed to a primer according to SEQ ID NO: 1-20 for use in a method of this invention. The precise sequence of SEQ ID NO: 1-20 can be found in Table 2. The numbering of SEQ ID NO: 1-20 corresponds to the order of the sequences indicated in the table.

Unless otherwise defined, all technical and scientific terms used herein have the same meaning as commonly understood by one of ordinary skill in the art to which this invention pertains. All publications, patent applications, patents, and other references mentioned herein are incorporated by reference in their entirety. In case of conflict, the present specification, including definitions, will control. In addition, the materials, methods, and examples are illustrative only and not intended to be limiting.

The invention is now further illustrated by the accompanying tables, which are showing the following:

Table 1 is showing elected characteristics of study subjects: Age, first degree family history, smoking habits etc.

Table 2 contains sequences of primers and masses of extension products of MALDI-TOF MS assays.

Table 3 shows the ERCC2 genotype frequencies in breast cancer cases and controls.

Table 4 illustrates combined genotypes of ERCC2_{—}6540_G>A (Asp312Asn) and ERCC2_{—}18880_A>C (Lys751Gln).

Table 5 shows haplotype frequencies of ERCC2_{—}6540_G>A (Asp312Asn) and ERCC2_{—}18880_A>C (Lys751Gln) in breast cancer cases and controls.

Table 6 illustrates ERCC2_{—}6540_G>A (Asp312Asn) genotypes and potential breast cancer risk factors.

### EXAMPLES

Materials and Methods

The GENICA Study Population Between August 2000 and October 2002 incidental breast cancer cases and population-based controls were recruited from the Greater Bonn Region in Germany, an area of more than 1 million inhabitants. This is part of a wider effort of the Interdisciplinary Study Group on Gene Environment Interactions and Breast Cancer in Germany (GENICA) which is focused on the identification of breast cancer risks. Works are therefore referred to as the GENICA study. There are 688 breast cancer cases with a first time diagnosis of primary breast cancer that was histologically confirmed within six months of enrolment, and 724 population-based controls matched in 5-year age classes. Details of the study population and recruitment procedures are given in Table 1 (Selected characteristics of study subjects: Age in 5-year classes, first degree family history, BMI, smoking habits). In brief, cases and controls were eligible if they were of Caucasian ethnicity, currently resided in the study region, and were below age 80 years. Risk factor information was collected via in-person interviews and included assessment of menopausal status, lifestyle factors, medical and family cancer history. All study participants provided a blood sample drawn into heparin tubes (Becton Dickinson green, No 367680, Franklin Lake, N.J., USA). The GENICA study was approved by the Ethic's Committee of the University Bonn and all study participants gave written informed consent.

Isolation of DNA Genomic DNA was extracted from 20 ml heparin blood samples using the Puregene™ (Gentra Systems, Inc., Mineapolis, USA) according to the manufacturer's instructions.

Genotyping Genotyping was performed at loci ERCC2_{—}6540_G>A and ERCC2_{—}18880 A>C (gDNA: Accession number L47234) as well as MTHFR_{—}677_C>T (mRNA: Accession number NM_{—}005957) and ABCB1_{—}3435_C>T (mRNA: Accession number NM_{—}000927) for genomic controls. Nucleotide positions were determined in gDNA sequence and in case of MTHFR and ABCB1 in mRNA starting with the A of the initial ATG as nucleotide 1.

Genotyping of single nucleotide polymorphisms (SNPs) was performed using the matrix assisted laser desorption/ionisation time-of-flight (MALDI-TOF) based mass spectrometry (MS) of allele specific primer extension products (Mass Array™, Sequenom, San Diego, Calif., USA). Briefly, 5 ng of genomic DNA was amplified by PCR in a final volume of 6 μl containing locus specific primers (Table 2) at 167 nM final concentrations and 0.1 unit HotStarTaq DNA Polymerase (Qiagen, Hilden, Germany). PCR conditions were 95° C. for 15 min for hot start, followed by 44 cycles of denaturation at 95° C. for 30 sec, annealing at 56° C. for 30 sec, and extension for 1 min at 72° C., finally followed by incubation at 72° C. for 10 min. PCR products were treated with shrimp alkaline phosphatase (SAP, Amersham, Freiburg, Germany) for 20 min at 37° C. to remove excess dNTPs followed by 10 min at 85° C. to inactivate SAP. Base extension [homogenous MassEXTEND (hME™), Sequenom, San Diego, Calif.] reactions in a final volume of 10 μl contained extension primers (Table 2) at a final concentration of 0.54 μM and 0.6 units ThermoSequenase (Amersham, Freiburg, Germany). Base extension reaction conditions were 94° C. for 2 min, followed by 40 cycles of 94° C. for 5 sec, 52° C. for 5 sec, and 72° C. for 5 sec. All reactions including PCR amplification, SAP treatment and base extension were carried out in a Tetrad PCR thermal cycler (MJ Research, Waltham, USA). The final base extension products, the fragment lengths of which are given in (Table 2) were treated with SpectroCLEAN resin (Sequenom, San Diego, Calif.; USA) to remove salts from the reaction buffer. This step was carried out with a Multimek 96-channel autopipette (Beckman Coulter, Fullerton, USA). For a final volume of 26 μl, 16 μl of resin-water suspension were added into each base extension reaction. Following a quick centrifugation (2,000 rpm, 3 min in an Eppendorf Centrifuge 5810, Hamburg, Germany), 10 nl of reaction solution was dispensed onto a 384 format SpectroCHIP microarray (Sequenom, San Diego, Calif.; USA) prespotted with a matrix of 3-hydroxypicolinic acid (3-HPA) by using a SpectroPoint nanodispenser (Sequenom, San Diego, Calif.; USA). A modified Bruker Biflex MALDI-TOF MS was used for data acquisitions from the SpectroCHIP. Genotyping calls were made in real time with MASSARRAY RT software v 3.0.0.4. (Sequenom, San Diego, Calif., USA).

Statistical Analysis Genotype frequencies were calculated and checked for Hardy Weinberg Equilibrium (HWE) according to Pearson. Odds ratios (OR) were calculated with the genotypes homozygous for rare alleles, i.e. ERCC2_{—}6540_AA and ERCC2_{—}18880_CC as reference, based on regular function in apoptosis (10, 26) and to facilitate comparisons (Zhu et al. 2004).

Haplotypes were estimated using PHASE (27, 28). Linkage disequilibrium (LD) was tested using Arlequin (http://lgb.unige.ch/arlequin).

Results

Genotype distributions of ERCC2_{—}6540_G>A and ERCC2_{—}18880_A>C were examined in cases and controls (Table 3). Among controls genotype frequencies at ERCC2_{—}6540 were 45% for GG, 42% for GA, and 13% for AA. Frequencies at ERCC2_{—}18880 were 41% for AA, 46% for AC, and 13% for CC. These frequencies were both in HWE.

Comparison of ERCC2_{—}6540_G>A genotype frequencies in patients and controls Our case population showed deviations from the control population in that there were 61% GG, 31% GA and 8% AA carriers. These observed frequencies were not in HWE (P<0.001). A comparison of frequencies between cases and controls identified the GG genotype as a significant breast cancer risk (OR=2.13, 95% CI: 1.44-3.16) (Table 3). This risk was significant for carriers of one or two G alleles (OR=1.67, 95% CI: 1.14-2.44) (Table 3).

Comparison of ERCC2_{—}18880_A>C genotype frequencies in patients and controls The observed frequencies were in HWE. No differences in genotype frequencies were observed between cases and controls (Table 3).

Combined genotype and haplotype frequencies The observed increased breast cancer risk of ERCC2_{—}6540_GG carriers was even higher when women were in addition carriers of the ERCC2_{—}18880_CC or AC genotype (OR=5.51, 95% CI: 2.37-12.81; OR=3.70, 95% CI: 2.09-6.53, respectively) (Table 4). When phase was established from genotypes by haplotype analysis we observed an association of the ERCC2_{—}6540_G/ERCC2_{—}18880_C haplotype with increased breast cancer risk (OR=3.30, 95% CI: 2.47-4.41) (Table 5).

When we estimated putative haplotype frequencies from allele frequencies we found that ERCC2_{—}6540_G/ERCC2_{—}18880_A and ERCC2_{—}6540_A/ERCC2_{—}18880_C haplotypes were more frequent than the risk associated ERCC2_{—}6540_G/ERCC2_{—}18880_C haplotype. This was confirmed by LD analyses that showed LD between the ERCC2_{—}6540_G and ERCC2_{—}18880_A allele as well as for the ERCC2_{—}6540_A allele and the ERCC2_{—}18880_C allele. This was true for both, cases and controls and highly significant (P<0.0001).

Genetic controls Integrity of our genotyping data was confirmed by independent evidence for matching genotype frequencies between the GENICA controls and published genotype frequencies in the Caucasian population with respect to non-DNA-repair related genes ABCB1 (formerly MDR1) and MTHFR.

Herein, an increased breast cancer risk for female carriers of the ERCC2_{—}6540_GG genotype in a German population is shown. Due to known multiple cellular ERCC2 functions it is important to view this observation within the various ERCC2 functional perspectives. ERCC2_{—}6540_GG encodes the frequent enzyme phenotype and carriers are predicted to have normal DNA repair proficiency. The NER aspect of ERCC2 function by itself therefore does not provide us with a rationale for the increased breast cancer risk. Rather, we may consider that ERCC2 is part of the basal transcription-repair complex TFIIH, a multi-subunit protein complex with multiple engagements (3, 29). Within this complex ERCC2 has been recognized as a member of the p53-mediated apoptotic pathway because TFIIH-associated ERCC2 binds p53, a key regulator of apoptosis of DNA damaged cells (30). This is corroborated by previous in vitro observations, in which primary human fibroblasts of individuals with ERCC2 germline mutations showed attenuated p53-mediated apoptosis. Moreover, following transformation of ERCC2 mutant fibroblasts with wild-type ERCC2 p53-mediated apoptosis was rescued (30). This TFIIH-p53-regulated apoptosis has been further linked with polymorphisms of ERCC2. In a comparison of the percentage of apoptotic lymphoblastoid cells detected 48 hours after UV radiation, cell lines homozygous for Asn at position 312 had 2.5 times more apoptotic cells than cells that were homozygous or heterozygous for Asp at position 312 (26). From these in vitro data we may speculate that carriers of the ERCC2_{—}6540_GG genotype, homozygous for Asp at position 312, may have a lower apoptotic capacity and be at increased risk to develop breast cancer. Conversely, the lower breast cancer risk of individuals homozygous for Asn at position 312 may be due to a higher apoptotic response. It is important to note however, that an in vitro model relevant to apoptosis and breast cancer would utilize an epithelial cell, inasmuch as apoptotic responses involve interactions with extracellular matrix substrate in contrast to lymphoblastoid cells (Danilkovitch et al. 2000). Thus, our data show that it would be of interest to compare the time course of apoptotic responses of different genotypes of adherent epithelial cells with the view that a subset of survivors of apoptotic response are potential cancer cells. Finally, our findings of an association of breast cancer risk and an evolutionarily highly conserved polymorphisms (10) stresses the role of a molecular evolutionary approach in the identification of breast cancer risks (Zhu et al 2004).

Due to its functional relevance we included the ERCC2_{—}18880_A>C polymorphism in our studies, since amino acid residue 751 is located within the interaction domain of ERCC2 and its helicase activator p44 protein inside TFIIH (7, 31). When looking for an association of breast cancer risk at this single locus we did not find any such association. However, combined genotype frequencies revealed that the observed ERCC2_{—}6540_GG (Asp312Asp) associated breast cancer risk increased significantly to more than 5-fold or 3-fold when women were also carriers of the ERCC2_{—}18880_CC (Gln751Gln) or ERCC2_{—}18880_AC (Lys751Gln) genotype, respectively. In particular, we identified the ERCC2_{—}6540_G/ERCC2_{—}18880_C (312Asp/751Gln) as the at-risk haplotype. With respect to calculated haplotype frequencies our data are reminiscent of that by Butkiewicz et al. (10) who suggested LD for codons 312 and 751. Similarly, our study suggests that carriers of 312Asp are most likely to be also carriers of 751Lys. Furthermore, our population-based controls revealed similar calculated haplotype frequencies when compared to those observed in segregation analysis of three-generation families by Butkiewicz et. al. (10). This may allow us to speculate that ERCC2 haplotype frequencies of the German and Polish populations are similar. In addition we compared the calculated haplotype frequencies and observed an imbalance in allelic combinations. This led us to estimate LD, which was highly significant. In particular, we observed an over representation of the 312Asn/751Gln and the 312Asp/751Lys haplotypes and under representation of the 312Asp/751Gln and 312Asn/751 Lys haplotypes. This imbalance was less pronounced for the 312Asn/751Gln haplotype in cases which establishes its role in breast cancer risk.

Although our data are in agreement with the functional role of ERCC2 and its polymorphisms in apoptosis control it is important to compare our results with those of others in order to point out consistencies and inconsistencies. ERCC2 polymorphisms have been subject to many cancer susceptibility studies, direct comparisons between studies however are frequently hampered by differences in ethnicities, organ sites, study size and controls. Interestingly, a hospital-based breast cancer case-control study of women from Korea did not find any breast cancer risk association with the ERCC2 Asp312Asn polymorphism (32). This discrepancy may be explained by different ethnic background in both studies, i.e. Asian versus Caucasian and/or differences in controls, i.e. hospital-based versus population-based. However, in a small lung cancer study, similar genotype risk and protection assignments were observed. The ERCC2 genotype encoding Asp312Asp was associated with an increased risk in light smokers whereas the 312Asn phenotype had a protective effect (10). In contrast, a large lung cancer study assigned the risk genotype to Asn312Asn in non- and mild smokers, but the same genotype was protective for heavy smoking individuals (17). Yet another lung cancer study did not find an association (4) which points to a critical role of study design. In prostate cancer, also the genotype encoding Asn312Asn has been recently assigned as the at-risk genotype within sibships and this risk was shown to further increase in combination with the XRCC1 genotype encoding Gln399Gln (22). XRCC1 operates in a different DNA repair pathway indicating that joint effects between genes of different DNA repair pathways may contribute to cancer risk. Aside from breast cancer and prostate cancer referring to gender specific conditions the prostate cancer study also differed with respect to the case-control design by using sibs i.e. cases and their brothers, which may explain a variation in risk allele assignment.

Overall, interpretations from our breast cancer study are in line with those by Butkiewicz et al. (10) for lung cancer but differ from other cancer studies in that we explain observed effects not by alterations in DNA repair capacity but rather by attenuated apoptosis. Also, in our study, potentially overriding stronger environmental risks were not considered for stratification into subgroups as it was the case in lung cancer studies were smoking exposure is considered a risk factor of higher magnitude than ERCC2 polymorphisms (17). Importantly, differences in the definition of risk genotypes/phenotypes may result from different risk mechanisms at different organ sites but also may reflect bias due to small study sizes or use of non-population-based case-control comparisons such as hospital-based controls, spouses and friends or members of the same sibship.

Our study benefits from sufficient large size and population-based design and our data are highly consistent with the functional interpretations derived from independent in vitro studies. Accordingly, the polymorphic ERCC2 acts as an intrinsic part of the organism's defense machinery by modulation of the p53 tumor suppressor function. Our analyses of clinical samples and controls support the concept that imbalances due to ERCC2 Asp312Asn and Lys751Gln polymorphism may contribute to breast cancer susceptibility by promoting the outgrowth of DNA-damaged breast epithelial cells. The origin of such DNA damage has not been subject to our study and therefore remains elusive. Yet, to our knowledge this is the first study assigning breast cancer risk to both the ERCC2 genotype encoding Asp312Asp and the haplotype encoding 312Asp/751Gln. In the future it will now be important to clarify the relevance of these ERCC2 polymorphisms in the prediction of breast cancer risk and prevention of the disease.

### REFERENCES

1. Pharoah, P. D., Antoniou, A., Bobrow, M., Zimmern, R. L., Easton, D. F., and Ponder, B. A. Polygenic susceptibility to breast cancer and implications for prevention. Nat Genet, 31: 33-6, 2002.
2. Helzlsouer, K. J., Harris, E. L., Parshad, R., Perry, H. R., Price, F. M., and Sanford, K. K. DNA repair proficiency: potential susceptiblity factor for breast cancer. J Natl Cancer Inst, 88: 754-5, 1996.
3. Hoeijmakers, J. H. Human nucleotide excision repair syndromes: molecular clues to unexpected intricacies. Eur J Cancer, 30A: 1912-21, 1994.
4. Spitz, M. R., Wu, X., Wang, Y., Wang, L. E., Shete, S., Amos, C. I., Guo, Z., Lei, L., Mohrenweiser, H., and Wei, Q. Modulation of nucleotide excision repair capacity by XPD polymorphisms in lung cancer patients. Cancer Res, 61: 1354-7, 2001.
5. Robles, A. I., Wang, X. W., and Harris, C. C. Drug-induced apoptosis is delayed and reduced in XPD lymphoblastoid cell lines: possible role of TFIIH in p53-mediated apoptotic cell death. Oncogene, 18: 4681-8, 1999.
6. Barnes, D. M., and Camplejohn, R. S. P53, apoptosis, and breast cancer. J Mammary Gland Biol Neoplasia, 1: 163-75, 1996.
7. Coin, F., Marinoni, J. C., Rodolfo, C., Fribourg, S., Pedrini, A. M., and Egly, J. M. Mutations in the XPD helicase gene result in XP and TTD phenotypes, preventing interaction between XPD and the p44 subunit of TFIIH. Nature Genet, 20: 184-188, 1998.
8. Eveno, E., Bourre, F., Quilliet, X., Chevallier-Lagente, O., Roza, L., Eker, A. P., Kleijer, W. J., Nikaido, O., Stefanini, M., Hoeijmakers, J. H., and et al. Different removal of ultraviolet photoproducts in genetically related xeroderma pigmentosum and trichothiodystrophy diseases. Cancer Res, 55: 4325-32, 1995.
9. Lunn, R. M., Helzlsouer, K. J., Parshad, R., Umbach, D. M., Harris, E. L., Sanford, K. K., and Bell, D. A. XPD polymorphisms: effects on DNA repair proficiency. Carcinogenesis, 21: 551-5, 2000.
10. Butkiewicz, D., Rusin, M., Enewold, L., Shields, P. G., Chorazy, M., and Harris, C. C. Genetic polymorphisms in DNA repair genes and risk of lung cancer. Carcinogenesis, 22: 593-7, 2001.
11. Caggana, M., Kilgallen, J., Conroy, J. M., Wiencke, J. K., Kelsey, K. T., Miike, R., Chen, P., and Wrensch, M. R. Associations between ERCC2 polymorphisms and gliomas. Cancer Epidemiol Biomarkers Prev, 10: 355-60, 2001.
12. Winsey, S. L., Haldar, N. A., Marsh, H. P., Bunce, M., Marshall, S. E., Harris, A. L., Wojnarowska, F., and Welsh, K. I. A variant within the DNA repair gene XRCC3 is associated with the development of melanoma skin cancer. Cancer Res, 60: 5612-6, 2000.
13. Vogel, U., Hedayati, M., Dybdahl, M., Grossman, L., and Nexo, B. A. Polymorphisms of the DNA repair gene XPD: correlations with risk of basal cell carcinoma revisited. Carcinogenesis, 22: 899-904, 2001.
14. Matullo, G., Guarrera, S., Carturan, S., Peluso, M., Malaveille, C., Davico, L., Piazza, A., and Vineis, P. DNA repair gene polymorphisms, bulky DNA adducts in white blood cells and bladder cancer in a case-control study. Int J Cancer, 92: 562-7, 2001.
15. Matullo, G., Palli, D., Peluso, M., Guarrera, S., Carturan, S., Celentano, E., Krogh, V., Munnia, A., Tumino, R., Polidoro, S., Piazza, A., and Vineis, P. XRCC1, XRCC3, XPD gene polymorphisms, smoking and (32)P-DNA adducts in a sample of healthy subjects. Carcinogenesis, 22: 1437-45, 2001.
16. Hou, S. M., Falt, S., Angelini, S., Yang, K., Nyberg, F., Lambert, B., and Hemminki, K. The XPD variant alleles are associated with increased aromatic DNA adduct level and lung cancer risk. Carcinogenesis, 23: 599-603, 2002.
17. Zhou, W., Liu, G., Miller, D. P., Thurston, S. W., Xu, L. L., Wain, J. C., Lynch, T. J., Su, L., and Christiani, D. C. Gene-environment interaction for the ERCC2 polymorphisms and cumulative cigarette smoking exposure in lung cancer. Cancer Res, 62: 1377-81, 2002.
18. Zhou, W., Liu, G., Miller, D. P., Thurston, S. W., Xu, L. L., Wain, J. C., Lynch, T. J., Su, L., and Christiani, D. C. Polymorphisms in the DNA repair genes XRCC1 and ERCC2, smoking, and lung cancer risk. Cancer Epidemiol Biomarkers Prev, 12: 359-65, 2003.
19. Park, J. Y., Lee, S. Y., Jeon, H. S., Park, S. H., Bae, N. C., Lee, E. B., Cha, S. I., Park, J. H., Kam, S., Kim, I. S., and Jung, T. H. Lys751Gln polymorphism in the DNA repair gene XPD and risk of primary lung cancer. Lung Cancer, 36: 15-6, 2002.
20. David-Beabes, G. L., Lunn, R. M., and London, S. J. No association between the XPD (Lys751Gln) polymorphism or the XRCC3 (Thr241Met) polymorphism and lung cancer risk. Cancer Epidemiol Biomarkers Prev, 10: 911-2, 2001.
21. Vogel, U., Laros, I., Jacobsen, N. R., Thomsen, B. L., Bak, H., Olsen, A., Bukowy, Z., Wallin, H., Overvad, K., Tjonneland, A., Nexo, B. A., and Raaschou-Nielsen, O. Two regions in chromosome 19q13.2-3 are associated with risk of lung cancer. Mutat Res, 546: 65-74, 2004.
22. Rybicki, B. A., Conti, D. V., Moreira, A., Cicek, M., Casey, G., and Witte, J. S. DNA repair gene XRCC1 and XPD polymorphisms and risk of prostate cancer. Cancer Epidemiol Biomarkers Prev, 13: 23-9, 2004.
23. Sturgis, E. M., Zheng, R., Li, L., Castillo, E. J., Eicher, S. A., Chen, M., Strom, S. S., Spitz, M. R., and Wei, Q. XPD/ERCC2 polymorphisms and risk of head and neck cancer: a case-control analysis. Carcinogenesis, 21: 2219-23, 2000.
24. Tang, D., Cho, S., Rundle, A., Chen, S., Phillips, D., Zhou, J., Hsu, Y., Schnabel, F., Estabrook, A., and Perera, F. P. Polymorphisms in the DNA repair enzyme XPD are associated with increased levels of PAH-DNA adducts in a case-control study of breast cancer. Breast Cancer Res Treat, 75: 159-66, 2002.
25. Goode, E. L., Ulrich, C. M., and Potter, J. D. Polymorphisms in DNA repair genes and associations with cancer risk. Cancer Epidemiol Biomarkers Prev, 11: 1513-30, 2002.
26. Seker, H., Butkiewicz, D., Bowman, E. D., Rusin, M., Hedayati, M., Grossman, L., and Harris, C. C. Functional significance of XPD polymorphic variants: attenuated apoptosis in human lymphoblastoid cells with the XPD 312 Asp/Asp genotype. Cancer Res, 61: 7430-4, 2001.
27. Stephens, M., and Donnelly, P. A comparison of bayesian methods for haplotype reconstruction from population genotype data. Am J Hum Genet, 73: 1162-9, 2003.
28. Stephens, M., Smith, N. J., and Donnelly, P. A new statistical method for haplotype reconstruction from population data. Am J Hum Genet, 68: 978-89, 2001.
29. Hoeijmakers, J. H., and Bootsma, D. Molecular genetics of eukaryotic DNA excision repair. Cancer Cells, 2: 311-20, 1990.
30. Wang, X. W., Vermeulen, W., Coursen, J. D., Gibson, M., Lupold, S. E., Forrester, K., Xu, G., Elmore, L., Yeh, H., Hoeijmakers, J. H., and Harris, C. C. The XPB and XPD DNA helicases are components of the p53-mediated apoptosis pathway. Genes Dev, 10: 1219-32, 1996.
31. Benhamou, S., and Sarasin, A. ERCC2/XPD gene polymorphisms and cancer risk. Mutagenesis, 17: 463-9, 2002.
32. Kang, D. Genetic polymorphisms and cancer susceptibility of breast cancer in Korean women. J Biochem Mol Biol, 36: 28-34, 2003.
33. Grainger, D. J., Heathcote, K., Chiano, M., Snieder, H., Kemp, P. R., Metcalfe, J. C., Carter, N. D., and Spector, T. D. Genetic control of the circulating concentration of transforming growth factor type beta1. Hum Mol Genet, 8: 93-7, 1999.

36. Siegsmund, M., Brinkmann, U., Schaffeler, E., Weirich, G., Schwab, M., Eichelbaum, M., Fritz, P., Burk, O., Decker, J., Alken, P., Rothenpieler, U., Kerb, R., Hoffmeyer, S., and Brauch, H. Association of the P-Glycoprotein Transporter MDR1(C3435T) Polymorphism with the Susceptibility to Renal Epithelial Tumors. J Am Soc Nephrol, 13: 1847-54, 2002.

TABLE 1
Characteristics of study participants

| | Cases, n (%) | Controls, n (%) |
|---|---|---|
| Age (y) | | |
| <50 | 140 (23) | 149 (23) |
| ≧50 | 468 (77) | 501 (77) |
| Menopausal status | | |
| Premenopausal | 146 (24) | 148 (23) |
| Postmenopausal | 455 (76) | 495 (77) |
| Smoking status | | |
| Never | 351 (58) | 362 (56) |
| Former | 139 (23) | 137 (21) |
| Current | 117 (19) | 151 (23) |
| Breast cancer in mother or sisters | | |
| No | 537 (88) | 601 (93) |
| Yes | 71 (12) | 49 (8) |
| Parity | | |
| 0 | 123 (20) | 120 (19) |
| ≧1 | 485 (80) | 529 (82) |
| HRT (y) | | |
| Never | 295 (49) | 327 (51) |
| >0 to <10 | 152 (25) | 188 (29) |
| ≧10 | 153 (26) | 130 (20) |

TABLE 2
Sequences of Primers and Masses of Extension Products of MALDI-TOF MS Assays

| | | | |
|---|---|---|---|
| SNP | Primer | Sequence | Mass (kDA) |

| | | | |
|---|---|---|---|
| ERCC2_6540_G > A | PCR-Primer 1 | 5′ ACG TTG GAT GTG CGA GGA GAC GCT ATC AGC 3′ | |
| | PCR-Primer 2 | 5′ ACG TTG GAT GAG TAC CGG CGT CTG GTG GAG 3′ | |
| | Extension Primer | 5′ CTC ACC CTG CAG CAC TTC GT 3′ | 5988.9 |
| | Analyte G | 5′ CTC ACC CTG CAG CAC TTC GTC 3′ | 6262.0 |
| | Analyle A | 5′ CTC ACC CTG CAG CAC TTC GTT G 3′ | 6606.0 |
| ERCC2_18880_A > C | PCR-Primer 1 | 5′ ACG TTG GAT GAG GAG CTA GAA TCA GAG GAG 3′ | |
| | PCR-Primer 2 | 5′ ACG TTG GAT GOA CCA GGA AGC GTT TAT GGC 3′ | |
| | Extension Primer | 5′ GAG CAA TCT GCT CTA TCC TCT 3′ | 6332.1 |
| | Analyte A | 5′ GAG CAA TCT GCT CTA TCC TCT T 3′ | 6620.3 |
| | Analyte C | 5′ GAG CAA TCT GCT CTA TCC TCT GC 3′ | 6934.5 |
| MTHFR_677_C > T | PCR-Primer 1 | 5′ ACG TTG GAT GCT TGA AGG AGA AGG TGT CTG 3′ | |
| | PCR-Primer 2 | 5′ ACG TTG GAT GCT TCA CAA AGO GGA AGA ATG 3′ | |
| | Extension Primer | 5′ GCT GCG TGA TGA TGA AAT CG 3′ | 6197.0 |
| | Analyte C | 5′ GCT GCG TGA TGA TGA AAT CGG C 3′ | 6799.4 |
| | Analyte T | 5′ GCT GCG TGA TGA TGA AAT CGA 3′ | 6494.2 |
| ABCB1_3435_C > T | PCR-Primer 1 | 5′ ACG TTG GAT GTG CCT ATG GAG AGA ACA GCC 3′ | |
| | PCR-Primer 2 | 5′ ACG TTG GAT GTA CAT TAG GCA GTG ACT CGA 3′ | |
| | Extension Primer | 5′ GGC CTC CTT TGC TGC GCT CAC 3′ | 6285.1 |
| | Analyte C | 5′ GGC CTC CTT TGC TGC CCT CAC GA 3′ | 6911.5 |
| | Analyte T | 5′ GGG CTC CTT TGC TGC CCT CAC A 3′ | 6582.3 |

TABLE 3
ERCC2 genotype frequencies in breast cancer cases and controls

| | Cases | Controls | | |
|---|---|---|---|---|
| Genotype | N (%) | N (%) | OR (95% Cl) | OR (95% Cl) |
| ERCC2_6540_G>A | | | | |
| (Asp312Asn) | | | | |
| AA | 47 (8) | 80 (13) | 1.00^{a} | 1.00^{a} |
| GA | 176 (31) | 257 (42) | 1.17 (0.78-1.75) | 1.67 (1.14-2.44)^{b} |
| GG | 347 (61) | 277 (45) | 2.13 (1.44-3.16) | |
| Allele | | | | |
| G | 270 (76) | 417 (66) | | |
| A | 870 (24) | 811 (34) | | |
| ERCC2_18880_A>C | | | | |
| (Lys751Gln) | | | | |
| CC | 101 (17) | 87 (13) | 1.00^{a} | 1.00^{a} |
| AC | 268 (45) | 295 (46) | 0.78 (0.56-1.09) | 0.76 (0.56-1.04)^{b} |
| AA | 227 (38) | 266 (41) | 0.74 (0.52-1.03) | |
| Allele | | | | |
| C | 470 (39) | 469 (36) | | |
| A | 722 (61) | 827 (64) | | |

^{b}Reference
^{a}Calculated from heterozygous and homozygous genotypes

TABLE 4
Combined genotypes of ERCC2_6540_G>A (Asp312Asn) and
ERCC2_18880_A>C (Lys751Gln)

| Genotypes | | Cases | | Controls | | |
|---|---|---|---|---|---|---|
| ERCC2_6540 | ERCC2_18880 | N (%) | | N (%) | | OR (95% Cl) |
| AA | CC | 29 | (5) | 47 | (8) | 1.00^{a} |
| AA | AC | 15 | (3) | 28 | (5) | 0.87 (0.40-1.89) |
| AA | AA | 3 | (1) | 5 | (1) | 0.97 (0.22-4.38) |
| GA | CC | 32 | (6) | 22 | (4) | 2.36 (1.16-4.81) |
| GA | AC | 118 | (21) | 199 | (33) | 0.96 (0.57-1.61) |
| GA | AA | 23 | (4) | 34 | (6) | 1.10 (0.54-2.22) |
| GG | CC | 34 | (6) | 10 | (2) | 5.51 (2.37-12.81) |
| GG | AC | 114 | (21) | 50 | (8) | 3.70 (2.09-6.53) |
| GG | AA | 189 | (34) | 218 | (36) | 1.41 (0.85-2.32) |

^{a}Reference

TABLE 5
Haplotype frequencies of ERCC2_6540_G > A (Asp312Asn) and
ERCC2_18880_A > C(Lys751Gln) in breast cancer cases and controls

| | | Cases | | Controls | | |
|---|---|---|---|---|---|---|
| ERCC2_6540 | ERCC2_18880 | N (%) | | N (%) | | OR (95% CI) |
| A | C | 216 | (19) | 332 | (27) | 1.00^{a} |
| A | A | 51 | (5) | 83 | (7) | 0.94 (0.64-1.39) |
| G | C | 221 | (20) | 103 | (8) | 3.30 (2.47-4.41) |
| G | A | 626 | (56) | 108 | (58) | 1.35(1.11-1.66) |

^{a}Reference

TABLE 6
ERCC2_6540_G > A (Asp³¹² Asn) genotypes and potential breast cancer risk factors

| AA | | | GA | | | GG | | |
|---|---|---|---|---|---|---|---|---|
| Cases, | Controls, | OR | Cases, | Controls, | OR | Cases, | Controls, | OR |
| n (%) | n (%) | (95% CI) | n (%) | n (%) | (95% CI) | n (%) | n (%) | (95% CI) |

Age (y)

| | | | | | | | | | |
|---|---|---|---|---|---|---|---|---|---|
| <50 | 15 (12) | 19 (13) | 1.00* (reference) | 29 (23) | 65 (46) | 0.59* (0.3-1.4) | 82 (65) | 57 (40) | 2.02* (0.9-4.5) |
| ≧50 | 32 (7) | 60 (13) | 1.00* (reference) | 144 (33) | 190 (41) | 1.44* (0.9-2.3) | 259 (60) | 219 (47) | 2.17* (1.4-3.5) |
| Menopausal status | | | | | | | | | |
| Premenopausal | 15 (12) | 17 (12) | 100* (reference) | 34 (27) | 65 (46) | 0.60* (0.3-1.4) | 81 (62) | 58 (41) | 1.56* (0.7-3.4) |
| Postmenopausal | 32 (8) | 62 (13) | 100* (reference) | 137 (32) | 185 (40) | 1.4* (0.9-2.3) | 255 (60) | 217 (47) | 2.20* (1.4-3.5) |
| Smoking status | | | | | | | | | |
| Never | 27 (8) | 42 (12) | 1.00^{†} (reference) | 105 (32) | 133 (39) | 1.20^{†} (0.7-2.1) | 195 (60) | 163 (48) | 1.77^{†} (1.0-3.0) |
| Ever | 20 (9) | 37 (14) | 1.00^{†} (reference) | 68 (29) | 122 (45) | 1.01^{†} (0.5-1.9) | 146 (62) | 113 (42) | 2.25^{†} (1.2-4.1) |

*OR_{adj} conditional on age in 5-year groups, adjusted for smoking, history of breast cancer in mother or sister, HRT, and parity.
^{†}OR_{adj} conditional on age in 5-year groups, adjusted for history of breast cancer in mother or sister, HRT, and parity.

1. A method of identifying an individual at an increased risk of breast cancer associated with a polymorphism in a gene, comprising
a) providing a sample from the individual containing genomic DNA;
b) determining the genotype of the ERCC2 gene of the individual;
c) identifying polymorphisms on the ERCC2 gene associated with the predisposition or susceptibility to breast cancer;
d) determining the predisposition or susceptibility to breast cancer of the individual.

2. The method of claim 1 wherein a polymorphism on the ERCC2 gene is at nucleotide position 6540.

3. The method of claim 1 or 2 wherein a polymorphism on the ERCC2 gene is at nucleotide position 1880.

4. The method of claim 2, wherein the polymorphism is AA, GA or GG.

5. The method of claim 3, wherein the polymorphism is CC, AC or AA.

6. The method of claim 4, wherein the predisposition or susceptibility to breast cancer is highest for GG.

7. The method of claim 4, wherein the predisposition or susceptibility to breast cancer is lowest for AA.

8. The method of one or more of claims 4, 6 and 7, wherein the predisposition or susceptibility to breast cancer is increasing in the following order: AA<GA<GG

9. The method of claim 5, wherein the predisposition or susceptibility to breast cancer is highest for CC.

10. The method of claim 4, wherein the predisposition or susceptibility to breast cancer is lowest for AA.

11. The method of claim 5, 9 or 10, wherein the predisposition or susceptibility to breast cancer is increasing in the following order: AA<AC<CC

12. The method of claim 1, wherein the predisposition or susceptibility to breast cancer is highest for: GG+CC, the first genotype being at nucleotide position 6540, the second at nucleotide position 18880.

13. The method of claim 1, wherein the predisposition or susceptibility to breast cancer is lowest for: AA+AC, the first genotype being at nucleotide position 6540, the second at nucleotide position 18880.

14. The method of claim 1, wherein the predisposition or susceptibility to breast cancer preferably is increasing in the following order: AA+AC<GA+AC<AA+AA<AA+CC<GA+AA<GG+AA<GA+CC<GG+AC<GG+CC, the first genotype of each pair being at nucleotide position 6540, the second at nucleotide position 18880.

15. A method of screening to identify compounds which modulate the apoptotic capacity of cells in an individual, comprising providing cells, in particular breast epithelial cells, of an individual genotype as defined in claim 1, contacting the cells with a candidate compound, observing the time course of apoptotic response of said cells and isolating those compounds, which are agonists of a higher apoptotic response in said cells.

16. An agonist identified by the method of claim 15.

17. An antibody, preferably a monoclonal antibody, directed against the amino acid sequence comprising the amino acid coded by the polymorphism on the ERCC2 gene at nucleotide position 6540 or 18880.

18. Primer according to SEQ ID NO: 1-20 for use in a method of claim 1.

19. A method of treating patients comprising identifying a patient with a predisposition to breast cancer by identifying polymorphisms in an ERCC2 gene associated with breast cancer according to claim 1 and administering to such patient an effective amount of an agonist of claim 16 in a pharmaceutically acceptable carrier.

20. Use of an agonist of claim 16 in a method of treating breast cancer.

21. Use of the antibody of claim 17 or a primer of claim 18 in the method of claim 1.


SEQUENCE LISTING (20 sequences)

SEQ ID NO: 1 - DNA, 30 bp, Artificial sequence
  Chemically synthesized PCR Primer 1 for ERCC2_6540_G>A
acgttggatgtgcgaggagacgctatcagc

SEQ ID NO: 2 - DNA, 30 bp, Artificial sequence
  Chemically synthesized PCR Primer 2 for ERCC2_6540_G>A
acgttggatgagtaccggcgtctggtggag

SEQ ID NO: 3 - DNA, 20 bp, Artificial sequence
  Chemically synthesized Extension Primer for ERCC2_6540_G>A
ctcaccctgcagcacttcgt

SEQ ID NO: 4 - DNA, 21 bp, Artificial sequence
  Chemically synthesized Analyte G for ERC C2_6540_G>A
ctcaccctgcagcacttcgtc

SEQ ID NO: 5 - DNA, 22 bp, Artificial sequence
  Chemically synthesized Analyte A for ERC C2_6540_G>A
ctcaccctgcagcacttcgttg

SEQ ID NO: 6 - DNA, 30 bp, Artificial sequence
  Chemically synthesized PCR Primer 1 for ERCC2_18880_A>C
acgttggatgagcagctagaatcagaggag

SEQ ID NO: 7 - DNA, 30 bp, Artificial sequence
  Chemically synthesized PCR Primer 2 for ERCC2_18880_A>C
acgttggatgcaccaggaaccgtttatggc

SEQ ID NO: 8 - DNA, 21 bp, Artificial sequence
  Chemically synthesized Extension Primer for ERCC2_18880_A>C
gagcaatctgctctatcctct

SEQ ID NO: 9 - DNA, 22 bp, Artificial sequence
  Chemically synthesized Analyte A for ERC C2_18880_A>C
gagcaatctgctctatcctctt

SEQ ID NO: 10 - DNA, 23 bp, Artificial sequence
  Chemically synthesized Analyte C for ERC C2_18880_A>C
gagcaatctgctctatcctctgc

SEQ ID NO: 11 - DNA, 30 bp, Artificial sequence
  Chemically synthesized PCR Primer 1 for MTHFR_677_C>T
acgttggatgcttgaaggagaaggtgtctg

SEQ ID NO: 12 - DNA, 30 bp, Artificial sequence
  Chemically synthesized PCR Primer 2 for MTHFR_677_C>T
acgttggatgcttcacaaagcggaagaatg

SEQ ID NO: 13 - DNA, 20 bp, Artificial sequence
  Chemically synthesized Extension Primer for MTHFR_677_C>T
gctgcgtgatgatgaaatcg

SEQ ID NO: 14 - DNA, 22 bp, Artificial sequence
  Chemically synthesized Analyte C for MTH FR_677_C>T
gctgcgtgatgatgaaatcggc

SEQ ID NO: 15 - DNA, 21 bp, Artificial sequence
  Chemically synthesized Analyte T for MTH FR_677_C>T
gctgcgtgatgatgaaatcga

SEQ ID NO: 16 - DNA, 30 bp, Artificial sequence
  Chemically synthesized PCR Primer 1 for ABCB1_3435_C>T
acgttggatgtgcctatggagacaacagcc

SEQ ID NO: 17 - DNA, 30 bp, Artificial sequence
  Chemically synthesized PCR Primer 2 for ABCB1_3435_C>T
acgttggatgtacattaggcagtgactcga

SEQ ID NO: 18 - DNA, 21 bp, Artificial sequence
  Chemically synthesized Extension Primer for ABCB1_3435_C>T
ggcctcctttgctgccctcac

SEQ ID NO: 19 - DNA, 23 bp, Artificial sequence
  Chemically synthesized Analyte C for ABC B1_3435_C>T
ggcctcctttgctgccctcacga

SEQ ID NO: 20 - DNA, 22 bp, Artificial sequence
  Chemically synthesized Analyte T for ABC B1_3435_C>T
ggcctcctttgctgccctcaca